(19) United States
(12) Patent Application Publication
(10) Pub. No.: US 2020/0038393 A1
(43) Pub. Date: Feb. 6, 2020

(54) APOMORPHINE PLUS BUSPIRONE COMBINATION FOR MEMORY REVIVAL

(71) Applicants: Huma Ikram, Karachi (PK); 2. Darakhshan Jabeen Haleem, Karachi (PK); Rumaisa Zakir, Karachi (PK)

(72) Inventors: Huma Ikram, Karachi (PK); 2. Darakhshan Jabeen Haleem, Karachi (PK); Rumaisa Zakir, Karachi (PK)

(21) Appl. No.: 16/053,596

(22) Filed: Aug. 2, 2018

(51) Int. Cl. A61K 31/485; A61K 31/506; A61P 25/28

(52) U.S. Cl. CPC A61K 31/485; A61P 25/28; A61K 31/506

(57) ABSTRACT

The invention introduces a method of inhibiting abuse potential and psychosis associated with long term use of apomorphine. Apomorphine used for the treatment of motor symptoms in Parkinson's disease produces addiction, and psychosis. The present method shows that apomorphine plus buspirone combination in the ratio of 1:1 will not produce psychosis. The combination will not be addictive too.

### BACKGROUND OF THE INVENTION

Non-addictive treatments for improving cognition are highly needed. Psychostimulants and other nootropic drugs used for improving cognition have abuse potential which limits their long term use. Apomorphine, having agonistic properties towards D1/D2 receptors, could be used in nondemented Parkinsonian patients to reduce amyloid-beta deposition. Thus, cognitive impairment in Parkinsonian patients could be reduced by apomorphine therapy. However, long term use of apomorphine produces psychosis and dependence.

Psychosis is modulated by multisynaptic descending pathways that include both dopaminergic as well as serotonergic mechanisms. Potentiating of reward delivery is predicted by release of dopamine in the striatum, which enhances the reward and responsiveness to conditioned stimuli. Apart from dopamine, serotonin also plays a crucial role in mediating the abusive potential of drugs. There is a well-established role of serotonin in not only establishment of drug use-associated behaviors, but also on the transition and maintenance of addictive effects of drugs.

As serotonergic system can modulate addictive effects of drugs of abuse. Co-administration of serotonergic agents is used to modulate the addictive effects of abusive drugs. It has been reported that hyperactivity produced by amphetamine, is reduced by the co-administration of 5HT precursor, 5-hydroxytryptamine.

There is an important role of 5HT-1A receptors in the addiction-related behaviors. It has been reported that mediation of behaviors related to addiction involve 5HT-1A autoreceptors. While expression of behaviors related to addiction is mediated by postsynaptic 5HT-1A receptors. Behavioral sensitization induced by Methamphetamine has also reported to be potentiated in 5HT-1A receptor knockout mice in both genders of rats.

It has been reported that repeated administration of apomorphine increases the sensitivity of somatodendritic 5-HT-1A receptors and apomorphine-induced behavioral sensitization (psychosis) is attenuated with the desensitization of these receptors.

Buspirone is an azaspirone drug and it is effective for the treatment of generalized anxiety disorders. It has agonistic affinity for 5HT-1A as well as antagonistic affinity for D2 receptors. Previously, we showed that buspirone plus apomorphine combination is non-addictive.

### BRIEF SUMMARY OF THE INVENTION

Apomorphine, could be considered as a novel therapeutic agent to treat cognitive impairment in Parkinson's/related disorders. However, a major limitation in using apomorphine as a therapeutic option, is its high abuse potential. Repetitive use of apomorphine produces sensitization, addiction and dependence. Serotonin has shown to play an important role in modulating addictive effects of drugs of abuse. 5HT-1A receptors are also supersensitized following repeated administration of apomorphine.

A method is therefore provided for inhibiting the addictive potential of apomorphine while keeping its therapeutic profile unaffected. Here we show that buspirone if co-administered with apomorphine could improve its therapeutic utility. Addiction to apomorphine is reduced and its memory enhancing effects are potentiated in buspirone co-injected animals. Moreover, psychosis like effect was not produced with this combination of apomorphine and buspirone.

### BRIEF DESCRIPTION OF THE SEVERAL VIEWS OF THE DRAWINGS

FIG. 1: Effects of apomorphine, buspirone and their co-administration on pre and post-conditioning values of time passed in the drug paired compartment and % age reinforcement values. Values are means±SD (n=6). Significant differences by Tukey's test: (A): *p<0.01 from respective pre-conditioning values; +p<0.01 from respective saline treated rats; # p<0.01 from respective 0 mg/kg buspirone treated rats following three way ANOVA. (B): *p<0.01 from respective saline injected rats; p<0.01 from respective 0 mg/kg buspirone treated rats following two-way ANOVA.

FIG. 2: Effects of apomorphine, buspirone and their co-administration on motor behavior post single- and repeated administration. Values are means±SD (n=6). Significant differences by Tukey's test: *p<0.01 from respective saline injected rats; +p<0.01 from respective 0 mg/kg buspirone injected rats following two way ANOVA.

FIG. 3: Effects of apomorphine, buspirone and their co-administration on memory acquisition, consolidation and retention as monitored in Morris water maze test. Values are means±SD (n=6). Significant differences by Tukey's test: *p<0.01 from respective saline injected rats; +p<0.01 from respective 0 mg/kg buspirone treated rats following two way ANOVA.

FIG. 4: Effects of apomorphine, buspirone and their co-administration on activities in novel environment of an open field. Values are means±SD (n=6). Significant differences by Tukey's test: *p<0.01 from respective saline injected rats; +p<0.01 from respective 0 mg/kg buspirone treated rats following two way ANOVA.

### DETAILED DESCRIPTION OF THE DRAWINGS

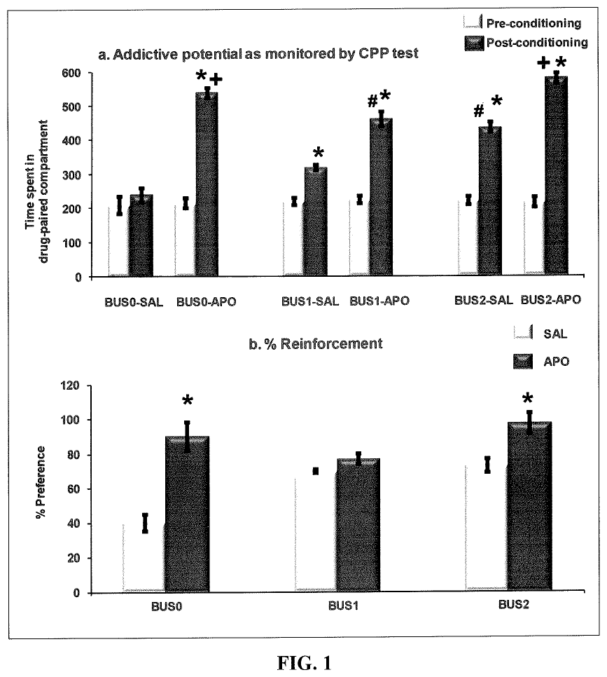

FIG. 1a shows results of conditioned place preference (CPP) test used to monitor addictive effects of apomorphine (1 mg/kg) in the absence or presence of buspirone (1 mg/kg and 2 mg/kg). Data analyzed by three-way ANOVA shows a significant effect of apomorphine (F=870.25 df1,60 p<0.0001), buspirone (F=141.17 df2,60 p<0.0001), and a significant interaction between apomorphine and buspirone (F=66.06 df2,60 p<0.0001). Repeated measure (pre and post injection monitoring) effects (F=401.56 df1,60 p<0.0001), repeated monitoringx apomorphine (F=841.00 df1,60 p<0.0001), repeated monitoring×buspirone (F=136.47 df2,60 p<0.0001) and repeated monitoring×buspironex morphine (F=1171.50 df2,60 p<0.0001) interactions are also significant.

Post hoc analysis by Tukey's test shows that pre-conditioning values of time spent in the drug paired compartment are comparable in the six groups. Post-conditioning values of time spent in the drug paired compartment are much higher in apomorphine treated animals suggesting repeated use of apomorphine is addictive. Co-administration of buspirone at the dose of 1 mg but not at the dose of 2 mg significantly attenuates apomorphine-induced increases in post conditioning values of time spent in drug paired compartment suggesting inhibition of the addictive effects of apomorphine in buspirone (1 mg/kg) co-treated animals.

Calculated percentage reinforcement values are shown in FIG. 1B. Two-way ANOVA shows effects of apomorphine (F=375.46 df1,30 p<0.0001), buspirone (F=120.29 df1,30 p<0.0001), and a significant interaction between apomorphine and buspirone (F=84.36 df1,30 p<0.0001) to be significant.

Post hoc analysis by Tukey's test shows that apomorphine injected at the dose of 1 mg/kg increased % reinforcement in all BUS0, BUS1 and BUS2 injected rats. This reinforcement effects did not occur if buspirone (1 mg/kg) is co-administered.

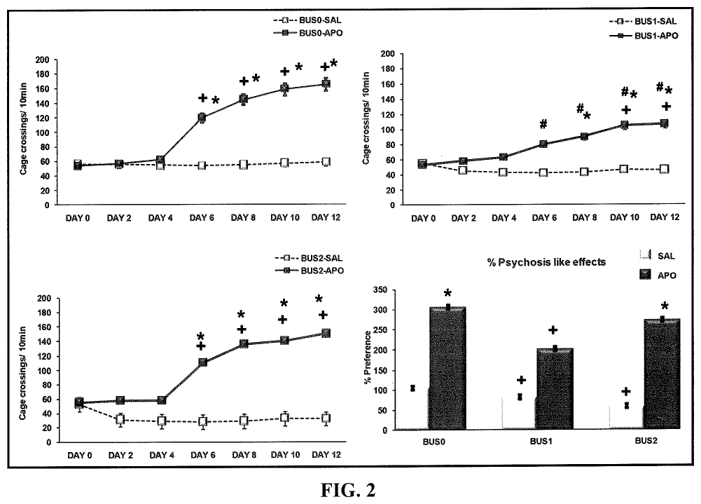

FIG. 2 depicts the effects of apomorphine, buspirone and their co-administration on motor behavior and percentage psychosis like effects. Two way ANOVA shows significant effect of apomorphine (F=368.35 df1,30 p<0.0001), buspirone (F=76.28 df1,30 p<0.0001) and interaction between apomorphine and buspirone (F=54.36 df1,30 p<0.0001) on motor activities post single administration. Effects of apomorphine (F=654.87 df1,30 p<0.0001), buspirone (F=87.29 df1,30 p<0.0001) and interaction between apomorphine and buspirone (F=43.28 df1,30 p<0.0001) on motor activities post repeated administration were also significant. FIG. 2c shows % psychosis like effects of apomorphine. Two way ANOVA shows significant effect of apomorphine (F=362.87 df1,30 p<0.0001), buspirone (F=53.28 df1,30 p<0.0001) and interaction between apomorphine and buspirone (F=42.29 df1,30 p<0.0001) on % psychosis like effects (FIG. 2c).

Post hoc analysis by Tukey's test shows increase in motor activities post single administration of apomorphine. Motor activities of activities were also increased post repeated administration of apomorphine but were attenuated in buspirone (1 mg/kg) co-treated rats. % psychosis like effects were produced by apomorphine but were attenuated in rats co-treated with buspirone at the dose of 1 mg/kg but not 2 mg/kg.

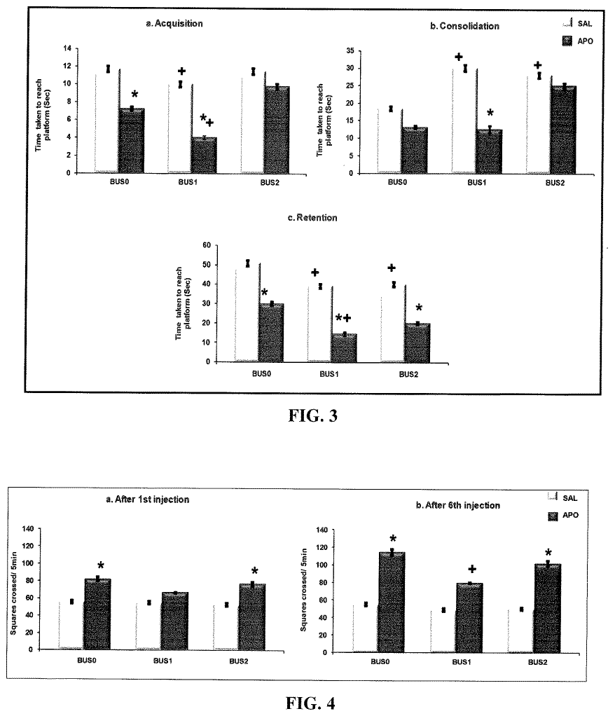

FIG. 3 depicts effect of different doses of apomorphine on learning acquisition, memory consolidation and retention as monitored in Morris water maze test. Two-way ANOVA shows significant apomorphine effects on learning acquisition (F=11.84 df1,30 p<0.001), memory consolidation (F=95.68 df1,30 p<0.001), and retention (F=816.50 df1,30 p<0.001). Effects of buspirone on learning acquisition (F=4.55 df2,30 p<0.01), memory consolidation (F=2.775 df2,30 p<0.05), and retention (F=116.03 df2,30 p<0.001) were all significant. Interaction between apomorphine and buspirone significantly affected learning acquisition (F=11.07 df1,30 p<0.001), memory consolidation (F=5.19 df1,30 p<0.01), and retention (F=3.46 df1,30 p<0.05). Post-hoc analysis shows an increase in learning acquisition, memory consolidation and retention as compared to saline treated animals (decreased time taken to reach platform). This was further potentiated in buspirone (1 mg/kg) but not buspirone (2 mg/kg) injected rats.

FIG. 4a depicts the effects of apomorphine, buspirone and their co-administration on exploratory activity in the novel environment of an open field after 1st apomorphine injection. Two way ANOVA (repeated measure design) shows significant effect of apomorphine (F=5.40 df1,30 p<0.01) buspirone (F=3.46 df2,30 p<0.05) and interaction between the two. (F=6.64 df2,30 p<0.05). Effects of apomorphine (F=147.20 df1,30 p<0.001) buspirone (F=12.21 df2,30 p<0.001) and interaction between the two (F=1.25 df2,30 p<0.01) were also significant as monitored in the open field after 6th apomorphine injection (FIG. 4b). Post hoc analysis by Tukey's test shows increased exploratory activity by apomorphine alone which was attenuated in buspirone (1 mg/kg) treated rats after 6th but not 1st apomorphine injection.

### DETAILED DESCRIPTION OF THE INVENTION

### Experiment

Co-administration of apomorphine with buspirone to study effects on apomorphine induced addiction, sensitization and memory enhancing effects.

Apparatus for Conditioned Place Preference

A three compartment conditioned place preference (CPP) apparatus with an unbiased design was used to monitor drug-induced reinforcement. The apparatus was made up of transparent plastic Perspex. The compartments were separated by sliding guillotine doors. The transparent middle (shuttle) compartment (14×26×26 cm) had a smooth floor and no stripes.

The end (preference) compartments (26×26×26 cm each) provided distinct contexts, with one compartment having black horizontal stripes on side walls, the other compartment had vertical black stripes. In this apparatus, animals showed no consistent preference for either compartment, which supported our unbiased CPP paradigm. The experiment was conducted in three distinct phases: pre-conditioning, conditioning and post-conditioning.

Pre-Conditioning Test

A pre-conditioning test ensured that the animals did not have a preference for any of the compartments. An animal introduced in the CPP apparatus from the middle compartment and guillotine doors were raised to open. The animal was allowed to explore the entire apparatus for 10 min. Time spent in each compartment was recorded.

Conditioning Phase

This phase started one day after pre-conditioning phase, animals were randomly assigned to six groups, each containing six animals: (i) buspirone (0 mg/kg)-saline, (ii) buspirone (0 mg/kg)-morphine, (iii) buspirone (1 mg/kg)-saline, (iv) buspirone (1 mg/kg)-apomorphine, (v) buspirone (2 mg/kg)-saline and (vi) buspirone (2 mg/kg)-apomorphine injected animals. Over next 12 days (day 1 to day 12) animals went through conditioning (one session per day) in which they were confined to either the horizontal or vertical stripe compartment by raising the respective guillotine door. On day 1, 3, 5, 7, 9 and 11 animals of all groups were injected with saline (1 ml/kg) and placed immediately in the assigned ‘Non-Drug paired’ compartment for 30 min. After which the animals were kept back in their home cages. On every other day, i.e. day 2, 4, 6, 8, 10, and 12 control and test animals were injected with saline, buspirone and/or apomorphine as assigned above. Immediately after injections, animals were placed in the ‘Drug paired’ compartment for 30 min. The animals picked from the drug paired compartment were placed back in their home cages.

Post-Conditioning Test

The test was carried out on day 13, 24 hr post last conditioning session, in a drug free state. Each animal was tested only once. As in the pre-conditioning phase, the guillotine doors were removed and rat was given access to the entire apparatus for 10 min. Time spent in ‘Drug paired’ compartment was monitored.

Motor Sensitization

Motor behavior was also monitored during conditioning phase. Animals confined to a compartment were moving across the compartment. Activity scores were counted as number of cage crossings for 10 min starting 5 min post injection.

Morris Water Maze Test

The Morris water maze test was conducted after repetitive apomorphine injections and post-conditioning. Water temperature was at room temperature, 21±1° C. The platform (10 cm×10 cm) was made of clear acrylic and was hidden 2 cm below the surface of water in a fixed location. Water was made opaque by adding milk to it.

Training

During training phase, each rat was placed into the water facing the wall of the tank and allowed 120 sec to locate and climb onto the submerged platform. The rat was allowed to stay on the platform for 10 sec. If it failed to find the platform within the allowed time it was guided gently onto the platform. After the training animals were tested three times.

Learning Acquisition

Each rat was tested immediately after training. Learning acquisition was determined by recording the initial latency (IL; the time taken by each rat to relocate the hidden platform).

Consolidation

Rats were tested for memory consolidation 60 min post training. It was determined by recording the retention latency (RL; the time taken by each rat to locate the hidden platform 1 h after training). The cut off time for each session was 2 min.

Memory Retention

Rats were tested for memory retention 24 hr post training session. Memory retention was determined by recording the retention latency (RL; the time taken by each rat to locate the hidden platform 24 hr post training). The cut off time for each session was 2 min.

Exploratory Behavior in Open Field

Open field consisted of a square area (76×76 cm) with walls 42 cm high. Floor of the apparatus was divided by lines into 25 squares of equal size. Experiment was conducted in a quiet room under white light. Animals were tested 24 hr post 1st and 6th apomorphine injection. Numbers of squares crossed with all four paws and latency to move from the central square were scored for 5 min.

1. A method of improving memory comprising administering to humans a suitable amount of a combination of apomorphine and buspirone.

2. The method of claim 1, wherein the said combination of apomorphine and buspirone is non-addicting.

3. The method of claim 1, wherein the memory loss is associated with Parkinsonism.

4. The method of claim 1, wherein buspirone prevents addiction to apomorphine through its serotonin-1A agonist properties.
